(19) United States
(12) Patent Application Publication
(10) Pub. No.: US 2013/0079370 A1
(43) Pub. Date: Mar. 28, 2013

(54) Method for the Inhibition of Deubiquitinating Activity

(71) Applicant: Vivolux AB, Uppsala (SE)

(72) Inventors: Slavica BRNJIC, Uppsala (SE); Padraig D'ARCY, Stockholm (SE); Rolf LARSSON, Uppsala (SE); Stig LINDER, Bromma (SE)

(73) Assignee: VIVOLUX AB, Uppsala (SE)

(21) Appl. No.: 13/623,415

(22) Filed: Sep. 20, 2012

(30) Foreign Application Priority Data
Sep. 23, 2011 (SE) 1100699-6

(51) Int. Cl. A61K 31/45

(52) U.S. Cl. CPC A61K 31/45
USPC 514/327

(57) ABSTRACT

A method of treating in a person a cancer tumor refractory to treatment with bortezomib or an agent sharing the apoptosis generating activity of bortezomib or any other anti-cancer drug, comprises administering to the person, in a pharmaceutically acceptable carrier, a pharmacologically effective dose of an agent selected from the group consisting of b-AP15 and other proteasome inhibitor abrogating the deubiquitinating (DUB) activity of the 19S RP DUBs.

### FIELD OF THE INVENTION

The invention relates to a method of treating cancer in a patient by inhibiting deubiquitinating activity. More particularly, the invention relates to a method of treating a cancer in a patient who has proved resistant to treatment by at least one anti-cancer medicine.

### BACKGROUND OF THE INVENTION

Tumor cells display enhanced sensitivity to disruptions in the ubiquitin-proteasome system (UPS) making this an attractive target for the development of anti-cancer therapies (1). Ubiquitin-tagged substrates are degraded by the 26S proteasome, a multi-subunit complex comprising a proteolytic 20S core (20S CP) capped by 19S regulatory particles (19S RP) (2,3). The 20S CP has evolved as an important target for anti-cancer drug development, resulting in the approval of bortezomib (Velcade®) for treatment of myeloic leukemia (4).

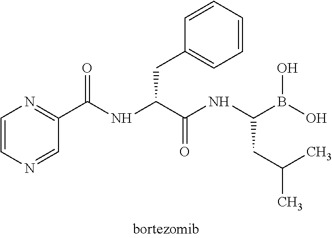

The compound b-AP15 (NSC687852) is known to induce p53-independent and cathepsin-D-dependent apoptosis (5,6).

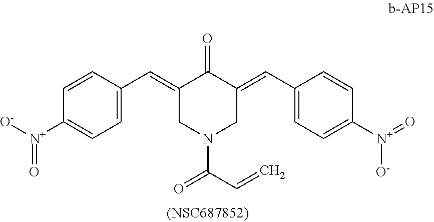 [SMILES: C=CC(=O)N1C/C(=C\c2ccc([N+](=O)[O-])cc2)C(=O)/C(=C/c2ccc([N+](=O)[O-])cc2)C1]

### OBJECTS OF THE INVENTION

It is an object of the invention to provide a method of treating cancer in a patient by inhibiting deubiquitinating activity.

Another object of the invention is to provide a method of treating cancer in a patient refractory to treatment with a deubiquitinating activity inhibiting medicine.

In particular, it is an object of the invention to provide a method of treating cancer in a patient refractory to treatment with at least bortezomib or an agent sharing the mechanism of deubiquitinating activitiy inhibition of bortezomib or any other kind of anti-cancer medicine.

Further objects of the invention will become evident by studying the following summary of the invention, a number of preferred embodiments thereof illustrated in a drawing, and the appended claims.

### SUMMARY OF THE INVENTION

According to the present invention the known compound b-AP15 is recognized as pertaining to a novel class of proteasome inhibitors that abrogate the deubiquitinating (DUB) activity of the 19S RP.

In particular, according to the present invention, b-AP15 inhibits the activity of two 19S RP DUBs, UCHL5 and USP14 while not affecting non-proteasomal DUBs.

Most particularly, according to the present invention, b-AP15 is effective in the treatment of a cancer refractory to treatment with bortezomib or an agent sharing the mechanism of deubiquitinating activity inhibition of bortezomib. In another preferred embodiment, b-AP15 is effective in the treatment of a cancer refractory to any anti-cancer drug.

In this application, “refractory to treatment” signifies that treatment of a cancer with a single dose of an anti-cancer medicine does not substantially reduce the growth rate of the cancer observed immediately prior to the treatment, such as reducing the growth rate per month by not more than 25 percent or 10 percent or even 5 percent or less. In particular, the method of the invention is efficient in treating a cancer in a patient which, after having received one or more, in particular two or three, standard doses of bortezomib or an agent sharing the apoptosis generating activity of bortezomib or any other anti-cancer drug, exhibits a cancer growth rate per month reduced by not more than 25 percent or 10 percent or even 5 percent or less, such as any positive growth rate, in comparison with the cancer growth rate observed immediately prior to the single treatment or to the last of two or three or more treatments, respectively. An accepted measure of tumor growth is the change of volume of a non-disseminated cancer.

An example of a cancer amenable to treatment by the method of the invention is multiple myeloma. Other examples of cancers amenable to treatment comprise lung cancer, prostate cancer, colon cancer, ovary cancer, pancreas cancer, breast cancer, neck & head cancer.

The method of the invention comprises administering to the patient in need a pharmacologically effective dose of b-AP15 in a suitable pharmaceutical carrier, such as, for instance, dissolved or suspended in an aqueous carrier or in a carrier comprising dimethyl sulfoxide or N,N-dimethylacetamide. Administration can be by any suitable route, such as by intravenous or intramuscular injection or infusion. Other methods of administration, in particular per os, are also contemplated, such as in form of tablets or gelatin capsules.

The person skilled in the art knows how to determine a pharmacologically effective dose. Such a dose may be from 0.01 g/kg body weight to 0.1 g or 1.0 g or more/kg body weight, consideration being given to whether the agent is administered systemically or locally.

Consistent with DUB inhibition, treatment with b-AP15 causes the accumulation of polyubiquitinated proteins of higher molecular weight in comparison with bortezomib treatment, and results in a stronger unfolded protein response. According to the invention, it has also been found that apoptosis induction by b-AP15 differs from that of bortezomib by being insensitive to disruption of the p53 tumor suppressor and insensitive to overexpression of the apoptosis inhibitor Bcl-2, BAX and PUMA.

According to the present invention treatment with b-AP15 inhibits tumor progression in human and mouse tumor in vivo models of breast, lung, colon, head & neck carcinoma, and inhibits infiltration in an acute myeloid leukaemia (AML) model. In consequence, inhibiting the DUB activity of the 19S RP by b-AP15 is disclosed to be a viable option for the treatment of cancer in humans and animals.

Thus, more specifically, is disclosed a method of treating in a person a cancer tumor refractory to treatment with bortezomib or with an agent sharing the apoptosis generating activity of bortezomib or with any other anti-cancer drug, comprising administering, in a pharmaceutically acceptable carrier, a pharmacologically effective dose of an agent selected from the group consisting of b-AP15 and other proteasome inhibitor abrogating the deubiquitinating (DUB) activity of the 19S RP DUBs. The method of the invention is particularly useful in the treatment of a patient having a tumor of which cells are refractory to treatment due to over-expression of the intrinsic apoptosis-inhibitor Blc-According to a preferred aspect of the invention the 19S RP DUBs comprise UCHL5 and USP14. According to another preferred aspect of the invention the deubiquitinating (DUB) activity of non-proteasomal DUBs is not affected by the method. The agent of the invention selected from the group consisting of b-AP15 and other proteasome inhibitor abrogating the deubiquitinating (DUB) activity of the 19S RP DUBs can be administered dissolved or suspended in a liquid carrier by any suitable route, such as by intravenous, intramuscular and subcutaneous administration. Alternatively or additionally, the agent selected from the group consisting of b-AP15 and other proteasome inhibitor abrogating the deubiquitinating (DUB) activity of the 19S RP DUBs can be administered perorally, such as in form of a tablet or capsule. A useful pharmacologically effective dose of the agent of the invention selected from the group consisting of b-AP15 and other proteasome inhibitor abrogating the deubiquitinating (DUB) activity of the 19S RP DUBs is from 0.01 g/kg body weight to 0.1 g or 1.0 g or more/kg body weight, consideration being given to whether the agent is administered systemically or locally. The method may comprise selecting a person to be treated by determining the growth rate of the cancer prior to and upon administration of bortezomib or said active principle sharing the mechanism of deubiquitinating activitiy inhibition of bortezomib or said other anti-cancer drug, a positive growth rate, in particular a growth rate of more than 5% or more than 10% or more than 25% per month constituting a selection marker.

The present invention comprises characterization of the functional connection between b-AP15 and other anti-cancer drugs by comparing the gene expression signature of b-AP15 treated cells with a collection of expression signatures for over 1300 bioactive compounds provided by the CMAP database (www.broad.mit.edu/cmap) (7). Treatment with b-AP15 induced a gene expression profile similar to that of several well characterized proteasome inhibitors, such as MG-262 (8), 15Δprostaglandin J2 (9), celastrol (10) and withaferin A (11).

b-AP15 blocks cellular proteasome function, as confirmed by use of a reporter cell line, which expresses ubiquitin tagged to yellow fluorescent protein (UbG76V-YFP) constitutively targeted for proteasomal degradation (12). Immunoblotting and flow cytometry revealed a dose dependent accumulation of the Ub-YFP reporter (IC50=0.8 μM) suggesting an impairment of proteasome function. Since inhibition of proteasome function is characterized by defects in ubiquitin turnover (13) colon carcinoma HCT116 cells were treated with b-AP15 and the level of ubiqutin conjugation analyzed by immunoblotting. The treatment caused the rapid time dependent accumulation of polyubiquitinated proteins of a higher molecular weight in comparison with the 20S CP inhibitor bortezomib, suggesting that b-AP15 inhibits an alternative branch of the UPS. The increase in polyubiquitin is associated with a strong proteotoxic response characterized by induction of HSPA6 (Hsp70B′), HSPA1B and DNAJB1 (Hsp40).

The turnover of many cell cycle regulatory proteins is controlled by the UPS including inhibitors of the cyclin-dependent kinase p21^{Cip1}, p27^{Kip1} and the tumor suppressor p53 (4).

Treatment with b-AP15 increased their levels in a dose dependent manner without altering the levels of ornithine decarboxylase 1 (ODC1), an ubiquitin-independent proteasome substrate (8). The increase in cell cycle regulators was concomitant with growth arrest in the G2/M phase boundary and increased sub G1 DNA content. The cell cycle arrest observed was not associated with increased levels of DNA damage markers such as phosphorylated p53 (at Ser 15) (9) or H2AX (at Ser 139) (10), suggesting that b-AP15 is not a genotoxic agent.

The increase in sub G1 DNA, caspase-3 activation and cleavage of poly-ADP ribose polymerase (PARP) and cytokeratin is associated with an overall decrease in cell viability at drug concentrations that induce the accumulation of polyubiquitin connecting UPS inhibition and apoptosis. Apoptosis induction by bortezomib is sensitive to the status of the p53 tumor suppressor and over-expression of the anti-apoptotic Bcl-2 oncoprotein (11, 12). By using isogenic clones of HCT116 colon cancer cells it was demonstrated that b-AP15 induced apoptosis is insensitive to over-expression of Bcl-2 and disruption of the apoptotic regulators p52, BAX or PUMA. Measurement of cytotoxic activity showed that b-AP15 is more toxic to the colon carcinoma cell line HTC-116 than to immortalized retinal pigment epithelial cells (hTERT-RPE1) and peripheral blood mononuclear cells (PBMC). b-AP15 exhibits a higher degree of cytotoxic activity towards the HTC-116 cells than to normal cell types.

The observed reduction in cellular proteasome activity cannot be explained by inhibition of proteolytic activities of the β subunits of the 20S CP. In vitro experiments using activity-specific substrates do not show inhibition in any of the proteolytic activities of the 20S CP or 26S proteasome, disassociation of the 19S RP and 20S CP or inhibition of polyubiquitin binding to the proteasome.

b-AP15 comprises an α-β dienone entity with two sterically accessible β carbons. A structurally similar pharmacophore has been earlier described to be comprised by a class of ubiquitin isopeptidase inhibitors (13). However, when cellular DUB activity was tested using ubiquitin 7-amido-4-methylcoumarin (Ub-AMC) on b-AP15 treated cells, no reduction in Ub-AMC cleavage could be observed. This demonstrates that b-AP15 is not a general DUB inhibitor. While not wishing to be bound by theory, the similarities in pharmacophore structure and the data showing that b-AP15 inhibits proteasome activity independent of the 20S CP indicate that b-AP15 inhibits the proteasome by blocking the deubiquitinating activity of the 19S RP.

In vitro assays using Ub-AMC and purified 19S RP or 26S proteasomes confirmed that b-AP15 inhibits the deubiquitinating activity of both the 19S RP and 26S proteasome. Recombinant ubiquitin-GFP is a substrate for 19S RP DUB activity (15). Treatment of 19S RP with b-AP15 efficiently inhibited the cleavage of Ub-GFP and ubiquinated HDM2. The type of ubiquitin bonds present in the polyubiquitin chain determines the fate of an ubiquitin-modified substrate.

K48 linked polyubiquitin chains generally target conjoined proteins for degradation (15), whereas K63 linked chains are involved in non-proteolytic roles including DNA repair (16) and mitotic chromosome segregation (17). Ubiquitin chain disassembly reactions revealed that b-AP15 inhibited 19S RP processing of both K48 and K63 linked ubiquitin tetramers. The inhibition of ubiquitin chain disassembly observed may account for the accumulation of high molecular weight ubiquitin conjugates in b-AP15 treated cells.

The deubiquitinating activity of the proteasome is attributed to the action of three DUBs, UCHL5, USP14 and POH1, all localized within the 19S RP (18-20). Both UCHL5 and USP14 are sensitive to N-ethylmaleimide (NEM), a general inhibitor of cysteine proteases, whereas POH1 is insensitive to inhibition by NEM but sensitive to metal chelators such as N,N,N,N-tetrakis-(2-pyridylmethyl)ethylenediamine (TPEN) (21). Inhibition experiments showed that residual DUB activity was present even after co-treatment of 19S RP with NEM and b-AP15. This residual DUB activity was abolished upon co-treatment of 19S RP with b-AP15 and TPEN, suggesting that b-AP15 primarily inhibits one or both of the NEM sensitive cysteine DUBs. The β-carbons in b-AP15 may serve as Michael acceptor moieties, resulting in covalent binding to cysteine residues in target proteins. In vitro assays showed, however, that b-AP15 is a reversible inhibitor and that glutathione does not preclude the inhibitory activity of the compound.

To identify specifically which DUBs were inhibited by b-AP15 treatment, competitive labelling experiments were performed using hemagglutinin tagged ubiquitin vinylsulphonone (HA-UbVS), an active site directed probe that irreversibly reacts with DUBs of the cysteine class (18). Incubation of 19S RP or 26S proteasomes with b-AP15 abolished Ub-VS labelling of two DUBs of molecular weights corresponding to UCHL5 and USP14. A similar result was obtained using UbVs on lysates derived from drug-treated cells. Immunoblot analysis showed a downward shift in molecular weight of both USP14 and UCHL5 due to loss of activity and decreased UbVs labelling. This is consistent with affinity-purified proteasomes from B-AP15 treated cells displaying reduced DUB activity confined to the proteasome and not evident in cell lysates. Additional in vitro assays showed minimal inhibition of b-AP15 on recombinant non-proteasomal cysteine DUBs, consistent with the notion that inhibition is not due to general cysteine reactivity.

b-AP15 does substantially decrease and even stop tumor growth in vivo, as shown by administration of b-AP15 to mice bearing either a human tumor or mouse xenografts. When b-AP15 was administered daily to SCID mice bearing FaDu head and neck carcinoma xenografts, significant inhibition of FaDu tumor growth was observed following daily treatment with b-AP15 (treated/control tumor volume, T/C=0.4, p=<0.001). Tumor cell death was analyzed by measuring xenograft derived cytokeratin (CK18) in circulation. Cytokeratin-18 is a biomarker for apoptosis (22, 23); a significant increase in plasma levels of total human CK18 was observed (p=0.01). Levels of caspase cleaved CK18 (CK18-Asp396) increased moderately compared with total levels, suggesting b-AP15 has activity against tumor cells in vivo. Also, b-AP15 was shown to inhibit tumor onset of HCT-116^{Bcl2+} colon carcinoma xenografts in nude mice, as shown by significant delay in tumor onset in comparison to vehicle treated controls; 2 out of 6 of the treated animals were completely disease free at the end of the study. Similarly, b-AP15 inhibits tumor growth in syngenic mice models using less frequent administration schedules.

Ubiquitin C-terminal hydrolases (UCH) and ubiquitin specific proteases (USP) are major subgroups of the approximately one hundred DUBs encoded by the human genome (24). The mechanism of specificity of b-AP15 for UCHL5 and USP14 in the 19S RP may be related to unique conformations of these enzymes in the 19S RP or due to drug-induced alterations of the 19S RP structure. The present findings are consistent with reports in the art indicating that loss of both UCHL5 and USP14, unlike loss of either one alone, leads to the accumulation of polyubiquitinated proteins and inhibition of cellular protein degradation (25).

The observation that DUB inhibition is associated with high molecular weight ubiquitin-substrate complexes seems to be of particular relevance. Strong expression of chaperone genes was observed in bAP15-treated cells, indicating induction of a proteotoxic response. High-molecular weight ubiquitin-substrate complexes accumulating as a result of DUB inhibition, such as by b-AP15 or a mechanistically equivalent compound, seem to generate strong cytotoxicity.

In the following the invention will be described in greater detail by reference to preferred embodiments thereof illustrated by a drawing comprising a number of figures.

### DESCRIPTION OF THE FIGURES

The figures of the drawing illustrate:

FIGS. 1a-1f Inhibition by b-AP15 of the ubiquitin-proteasome system;

FIGS. 2a-2e Inhibition b-AP15 of deubiquitination by the 19S RP;

FIGS. 3a-3d Inhibition by b-AP15 of the 19S RP DUBs UCHL5 and USP14;

FIGS. 4a-4h Inhibition by b-AP15 of tumor growth in vivo;

FIG. 5 Absence of DNA damage potentially caused by b-AP15;

FIGS. 6a-6f Induction by b-AP-15 of apoptosis and inhibition of cell survival of HCT-116 cells;

FIG. 7 Dose response curves of apoptosis induction in isogenic clones of HCT-116 cells;

FIGS. 9a, 9b Absence of dissociation of 19S and 20S particles potentially caused by b-AP15 or alteration of ubiquitin binding caused by b-AP15;

FIG. 10 b-AP15 not being a general DUB inhibitor;

FIGS. 11a-11c Biochemical characterization of b-AP15 binding;

FIGS. 12a-12c b-AP15 not being a general DUB inhibitor;

FIG. 13 Absence of significant alteration of animal weight by treatment with b-AP15;

FIG. 14 Sensitivity of cell lines in the NC160 cell line to b-AP15 and bortezomib.

### DESCRIPTION OF PREFERRED EMBODIMENTS

### Methods

In vitro proteasome activity assays were performed in black 96-well microtitier plates using human 20S proteasome (Boston Biochem) in reaction buffer (25 mM Hepes, 0.5 mM EDTA, 0.03% SDS) with Suc-LLVY-AMC, Z-LLE-AMC or Boc-LRRAMC used as substrates for proteasome activity. De-ubiquitinase activity assays were performed with human 19S RP (Boston Biochem) with ubiquitin-AMC as substrate. For FaDu xenograft studies a 100-μl-cell suspension containing 1×10⁶ cells was injected subcutaneously into the flank of SCID. Upon tumor take mice were randomized into control or treatment groups and administered with 5 mg kg⁻¹ b-AP15 or vehicle. In vivo levels of apoptosis and cell death were determined from the detection of caspase cleaved and total levels of cytokeratin-18 in plasma using M30 Apoptosense® and M65 ELISA®s assays (Peviva). The methods are described below in more detail.

Reagents.

Reagents were obtained from the following sources: 20S proteasome (E-360), 26S proteasome (E-365), 19S proteasome (E-366), Suc-LLVY-AMC (S-280), Z-LLE-AMC (S-230), Boc-LRR-AMC (S-300), Ubiquitin-AMC (U-550), Tetra-ubiquitin K63 (UC-310), Tetra-ubiquitin K48 (UC-210), deconjugating enzyme set (KE10), HA-Ubiquitin Vinyl Sulfone (U-212) (Boston Biochem); anti-β-actin (AC-15), ODC-1 (HPA001536) (Sigma Aldrich); anti-LC-3 (2775), anti-GAPDH (2118), anti-p44/42 MAPK (4695), anti-Phospho-p44/42 MAPK (9101) (Cell Signaling); N-ethylmaleimide (34115) (EMD Chemicals); anti-Ubiquitin K48 (Apu2), anti-Ubiquitin (MAB1510) (Millipore); anti-p53 (D01), anti-UCHL5 (H-110), Hdm2 (SMP14) (Santa Cruz); anti-PARP (C2-10), anti-p27 (G173-524), anti-active Caspase 3 (C92-605) (BD Biosciences); anti-USP14 (A300-919A) (Bethyl Laboratories); anti-HA (12CA5) (Roche); b-AP15 (NSC687852) was obtained from the Developmental Therapeutics Program of the US National Cancer Institute (http://www.dtp.nci.nih.gov) or synthesized by OncoTargeting AB (Uppsala, Sweden). Bortezomib was obtained from the Department of Oncology, Karolinska Hospital, Sweden.

Cell Culture.

MCF7 cells were maintained in MEM/10% fetal calf serum. HCT-116 p53+/+, p53−/−, Bcl-2+/+, PUMA−/− and BAX−/− cells were maintained in McCoy's 5A modified medium/10% fetal calf serum. The HCT-116 p53+/+, p53−/−, PUMA−/− and BAX−/− were generated as described (36). The HCT-116 Bcl-2+/+ cell line was generated by transfecting parental HCT-116 p53+/+ cells with pCEP4 Bcl-2 (Addgene plasmid 16461) (37) and isolating high expression clones. FaDu and LLC3 cells were maintained in DMEM high glucose medium supplemented with 10% fetal calf serum, Na pyruvate, Hepes and non-essential amino acids. 4T1.12B carcinoma cells were maintained in RPMI medium supplemented with 10% fetal calf serum. The proteasome reporter cell line MelJuSo Ub-YFP was generated as described (38). Cells were maintained in Dulbecco's Modified Eagle's Medium/10% fetal calf serum. The retinal epithelial cell line was generated as described (39). All cells were maintained at 37° C. in 5% CO₂.

Connectivity Map Analysis.

The microarray based gene expression analysis and the Connectivity Map (CMAP) analysis was performed as previously described (40). Briefly, MCF7 cells were exposed to b-AP15 (1 μM, 6 h) or vehicle (0.1% DMSO, 6 h). RNA was isolated (RNeasy miniprep kit, Qiagen) followed by quality control, labelling and hybridization to Genome U133 Plus 2.0 arrays (Affymetrix Inc). Raw data was normalized using MasS (Affymetrix Inc.) and rank ordered. For selection of the 30 most induced (up tags) and the 30 most suppressed (down tags) transcripts the following criteria were used: Up tags, present call and expression over 300 arbitrary in the b-AP15 experiment; Down tags, present call after both b-AP15 and vehicle treatment, and expression over 300 arbitrary units in the vehicle experiment. For CMAP compatibility only tags (i.e. probes) present on HG U133A were used. Raw and normalized expression data have been deposited at Gene Expression Omnibus (http://www.ncbi.nlm. nih.gov/geo/) with accession number GSE24150.

Proteasome and DUB Inhibition Assays.

In vitro proteasome activity assays using 20S CP (2 nM) (Boston Biochem) were performed at 37° C. in 100-μl reaction buffer (25 mM Hepes, 0.5 mM EDTA, 0.03% SDS). Samples were incubated for 10 min with indicated compound followed by addition of 10 μM Suc-LLVY-AMC, Z-LLE-AMC or Boc-LRR-AMC for the detection of chymotrypsin-like, caspase-like and trypsin-like activity respectively. For DUB inhibition assays 19S RP (5 nM), 26S (5 nM) UCH-L1 (5 nM), UCH-L3 (0.3 nM), USP2CD (5 nM) USP7CD (5 nM) USP8CD (5 nM) and BAP1 (5 nM) were incubated with b-AP15 followed by addition of ubiquitin-AMC (1000 nM). Fluorescence was monitored using Wallac Multilabel counter or Tecan Infinite M1000 equipped with 360 nm excitation and 460 nm emission filters. Substrate overlay assays. Native gel electrophoresis was performed as described (41). In brief 4 μg of purified 26S proteasome (Boston Biochem) was mixed with 10 or 50 μM b-AP15 and incubated at 37° C. for 10 min. Samples were resolved on 4% non-denaturing PAGE. Gels were submerged in assay buffer (20 mM Tris-HCL, 5 mM MgCl₂, 1 mM ATP, 0.1 mM Suc-LLVY-AMC) and proteasomes were visualized under UV illumination.

Ubiquitin-Cleavage Assay.

The recombinant Ub-GFP plasmid pet19b Ub-M-GFP was generated as described (42). In brief recombinant Ub-GFP was purified from BL21 E. coli cells by His affinity purification. For cleavage assays 19S RP (25 nM) was incubated with 10 mM NEM, 250 μM TPEN or 50 μM b-AP15 for 10 min followed by the addition of recombinant Ub-GFP (200 nM). Ubiquitin chain disassembly reactions were performed essentially as above except K48- or K63-linked ubiquitin tetramers (50 ng) were substituted for Ub-GFP. The level of Ub-GFP cleavage or ubiquitin disassembly was determined by immunoblotting with anti ubiquitin antibodies. The ubiquitinated Hdm2 substrate was generated according to the Boston Biochem protocol (K-200). For the cleavage assay 19S RP (25 nM) was incubated with 50 μM b-AP15 or DMSO for 10 min followed by the addition of ubiquitinated Hdm2 substrate (100 nM). The cleavage of ubiquitinated Hdm2 substrate and ubiquitinated Hdm2 was determined by immunoblotting with anti-Hdm2 antibodies.

Proteasome Isolation:

HCT-116 cells were treated with bortezomib (100 nM) or b-AP15 (1 μM) for 3 hours. After stimulation, the cells were lysed in 50 mM HEPES pH 7.4, 250 mM sucrose, 10 mM MgCl₂, 2 mM ATP, 1 mM DTT and 0.025% digitonin. Samples were sonicated briefly and incubated for 15 min on ice. Proteasomes from these samples were isolated according to the manufacturer's protocol.

UbVS Labelling of DUBs.

For labelling of DUBs in cell lysates sub confluent cells were harvested by trypsinization, washed three times with PBS, and centrifuged at 1500 RPM for 5 min. Cell pellets were lysed with buffer (50 mM HEPES pH 7.4, 250 mM sucrose, 10 mM MgCl₂, 2 mM ATP, 1 mM DTT) on ice for 15 min. Debris was removed by centrifugation and 25 μg of protein was labelled with 1 μM HA-UbVS for 30 min at 37° C. Samples were resolved by SDS-PAGE and analyzed by immunoblotting with indicated antibodies.

Determination of Cell Apoptosis and Viability.

For determination of apoptosis parental HCT-116 p53+/+ cells were treated with the increasing doses of bortezomib or b-AP15 for 24 h. Treatment doses were based on the drug concentration that resulted in maximal apoptosis over a 24 h period. HCT-116 cells were seeded in 96-well microtiter plates at 10,000 cells per well and incubated overnight. Cells were treated with indicated drug for 24 h. At the end of the incubation period, NP40 was added to the tissue culture medium to 0.1% and 25 μl of the content of each well was assayed using the M30-Apoptosense® ELISA as previously described (43). Cell viability was determined by measuring acid phosphatase activity or using the FMCA method (44). For the acid phosphatase activity cells were seeded at 5000 cells per well in 96-well culture plates and incubated for 12 h at 37° C. Compounds were added to the cells in growth media and incubated for 72 h at 37° C. Cells were washed with 200 μl warm PBS. 100 μl of para-nitrophenyl phosphate (pNPP, 2 mg/ml) in Na acetate buffer pH 5 (NaAc 0.1 M, 0.1% Triton-X-100) was added per well. Cells were incubated for 2 h after which reaction was stopped by addition of 1N NaOH. Absorbance was measured at 405 nm.

For the FMCA assay cells were seeded in the drug-prepared 384-well plates using the pipetting robot Precision 2000 (Bio-Tek Instruments Inc., Winooski, Vt.). The plates were incubated for 72 h and then transferred to an integrated HTS SAIGAN Core System consisting of an ORCA robot (Beckman Coulter) with CO₂ incubator (Cytomat 2C, Kendro, Sollentuna, Sweden), dispenser module (Multidrop 384, Titertek, Huntsville, Ala.), washer module (ELx 405, Bio-Tek Instruments Inc), delidding station, plate hotels, barcode reader (Beckman Coulter), liquid handler (Biomek 2000, Beckman Coulter) and a multipurpose reader (FLUOstar Optima, BMG Labtech GmbH, Offenburg, Germany) for automated FMCA. Survival index (SI) is defined as the fluorescence of test wells in percentage of controls with blank values subtracted.

Cell-Cycle Analysis.

For determination of cell cycle HCT-116 cells were treated with b-AP15 or DMSO Cells were harvested by trypsinisation, washed and fixed in 70% ice cold EtOH for 12 h. Cells were re-suspended in staining solution containing propidium iodide (50 μg/ml) and RNAse A (0.5 μg/ml) in PBS. Samples were run on BD FACScalibur. The percentage of cells in each phase of the cell cycle was determined using ModFit software.

In Vivo Tumor Experiments.

Animal experiments were conducted in full accordance with Swedish governmental statutory regulations on animal welfare under permission from local ethical committees. Animals were housed at a max of five per cage and provided with sterile water and food ad libitum. All mice were monitored and weighed daily. For the head and neck carcinoma model a 100-μl cell suspension containing 1×10⁶ FaDu cells was injected subcutaneously into the right rear flank of the animals. After injection, tumor growth was measured daily with calipers and the tumor volume calculated by the formula L×W²×0.44. When tumors had grown to a size of approximately 200 mm³ (Day 0) mice were randomized to receive either vehicle (n=10) or b-AP15 5 mg/kg by subcutaneous injection s.c. (n=15) daily. For the colon carcinoma model, 2.5×10⁶ HCT-116 colon carcinoma cells stably transfected with Bcl-2 (HCT-116^{Bcl-2+}) were inoculated subcutaneously into the right flank of nude mice. One day after inoculation mice were treated with 5 mg/kg⁻¹ by intra peritoneal injection (i.p.). Animals were inspected daily to establish the tumor onset and growth. For the lung carcinoma model a 100-μl cell suspension containing 2×10⁵ Lewis Lung Carcinoma (LLC) cells was injected subcutaneously into the right rear flank of C57/B6 mice. When tumors had grown to a size of approximately 50 mm³ (Day 0) mice were randomized to receive either vehicle (n=4) or b-AP15 5 mg/kg⁻¹ i.p. (n=4) with a treatment cycle consisting of two days treatment followed by two days no treatment (2 days on/2 days off) for two weeks. For the breast carcinoma model a 100-μl cell suspension containing 1×10⁵ 4TD cells was injected subcutaneously into the right mammary fat pad of BALB/c mice. When tumors had grown to a size approximately 25 mm³ (Day 0), mice were randomized to receive either vehicle (n=5) or b-AP15 2.5 mg/kg⁻¹ i.p. (n=5) with a treatment cycle consisting of one days treatment followed by three days no treatment (1 day on/3 days off) for 3 weeks. In the AML studies female C57BL/6J mice were injected i.v. in the tail vein with 5×10⁵ C1498 AML cells. After eight days mice were randomized to receive either b-AP15 5 mg/kg⁻¹ (n=10) or vehicle (n=10) i.p. for 7 days (day +8 till +14). Nineteen days after malignant cell injection all of the mice were killed and histopathological manifestations of liver, ovary (target organs for this model of tumor) were evaluated and compared between groups. For administration of drug b-AP15 was dissolved in Cremphor EL:PEG 400 (1:1) by heating to give a working concentration of 2 mg/ml. Working stock was 1:10 diluted in 0.9% normal saline immediately prior to injection.

Determination of Caspase-Cleaved CK18 in Mouse Plasma.

For measurement of the apoptosis-related CK18-Asp396 fragment, 12.5 ml of plasma was collected 24 h after last treatment and analyzed using the M30-Apoptosense® assay. Each sample was mixed with 0.4 ml of heterophilic blocking reagent (Scantibodies Laboratory Inc).

Determination of Pulmonary Metastases.

Since the 4T1 cells are resistant to 6-thioguanine, metastases can be determined by culturing homogenized tissue in the presence of 6-thioguanine. For determination of metastastic 4T1 cells the protocol was as described (45). In brief lungs from treated or untreated animals were homogenized and treated with collagenase and elastase. Cells were grown in the presence of 60 μM 6-thioguanine for 2 weeks and the number of metastatic colonies determined by giemsa staining.

Immunostaining.

Tumor sections were de-paraffinized with xylene, rehydrated and then incubated over-night with K-48 ubiquitin or active-caspase 3 (1/500) diluted in 1% (wt/vol) bovine serum albumin and visualized by standard avidin-biotin-peroxidase complex technique (Vector Laboratories). Counterstaining was performed with Mayer's haematoxylin.

Statistical Analyses.

For comparisons of treatment groups, we performed the unpaired t test (Mann-Whitney), repeated measures ANOVA and Kaplan-Meier survival (Mantel-Cox test). All statistical analyses were performed using GraphPad Prism Software (version 5.0). Statistical significance was achieved when P was less than 0.05.

### Example 1

### b-AP15 Inhibits the Ubiquitin-Proteasome System

CMAP readout of MCF7 cells treated with b-AP15 (1 μM) for 6 h is shown in Table 3.

TABLE 3
CMAP readout of MCF7 cells treated with B-AP15

| Rank | Name | Cell | Score |
|---|---|---|---|
| 1 | MG-262 | MCF7 | 1 |
| 2 | 15 Δ prostaglandin J2 | ″ | 0.999 |
| 3 | Celastrol | ″ | 0.966 |
| 4 | 15 Δ prostaglandin J2 | ″ | 0.940 |
| 5 | Withaferin A | ″ | 0.922 |

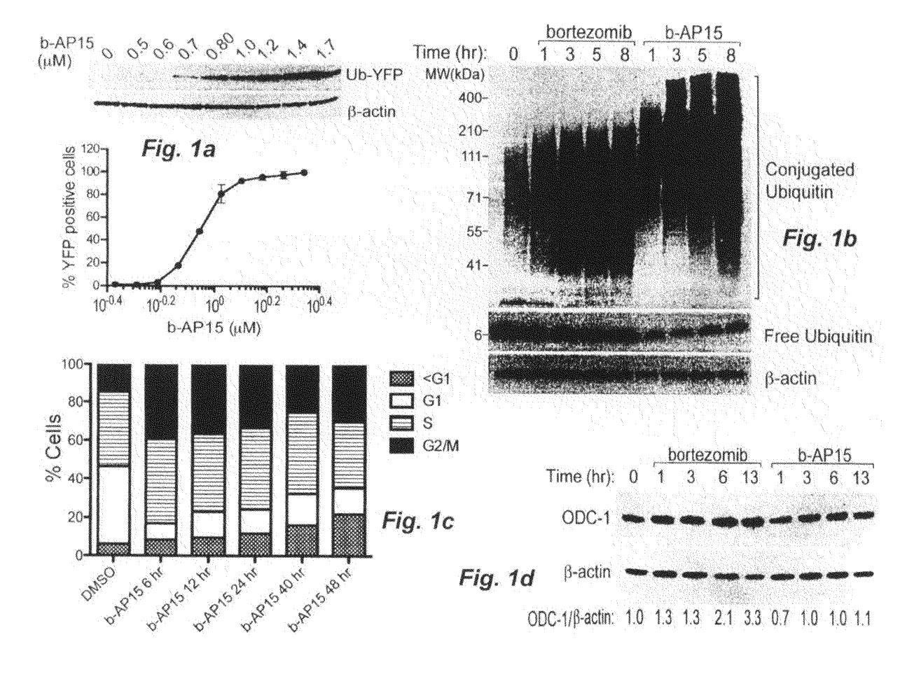

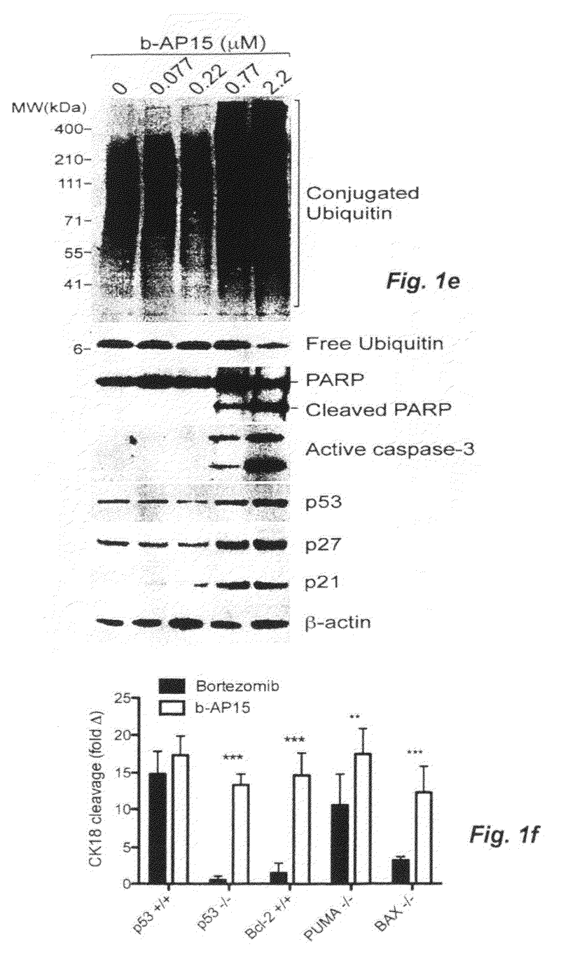

b-AP15 inhibits degradation of ubiquitin-tagged YFP in a proteasome reporter cell line (FIG. 1a). Levels of ^{UbG76V}-YFP accumulation were determined by flow cytometry and immunoblotting; immunoblot of ubiquitin conjugation in HCT-116 cells treated with b-AP15 (1 μM) or bortezomib (100 nM) (FIG. 1b). Immunoblot of ubiquitin conjugates, caspase 3 activation PARP cleavage, p53. P21^{Cip1} and p^{Kip1} in HCT-116 cells following 24 h treatment with the indicated concentrations of b-AP15 (FIG. 1c). Immunoblot of ODC-1 levels in HCT-116 cells following treatment with bortezomib (100 mM or b-Ap15 (1 μM) (FIG. 1d); values represent quantified optical density units of ODC-1 normalized to β-actin. Cell cycle profiles of b-AP15 treated HCT-116 cells (FIG. 1e); cells were analyzed by propidium iodide staining and flow cytometry. Levels of caspase activity in isogenic HCT-116 cells as determined by ELISA for caspase cleaved cytokeratin-18 (CK18-Asp398) following treatment with bortezomib (100 nM) or b-AP15 (1 μM) (**′P=0.01, ***′P=0.001) (FIG. 1f).

### Example 2

### b-AP15 Inhibits Deubiquitination by the 19S RP

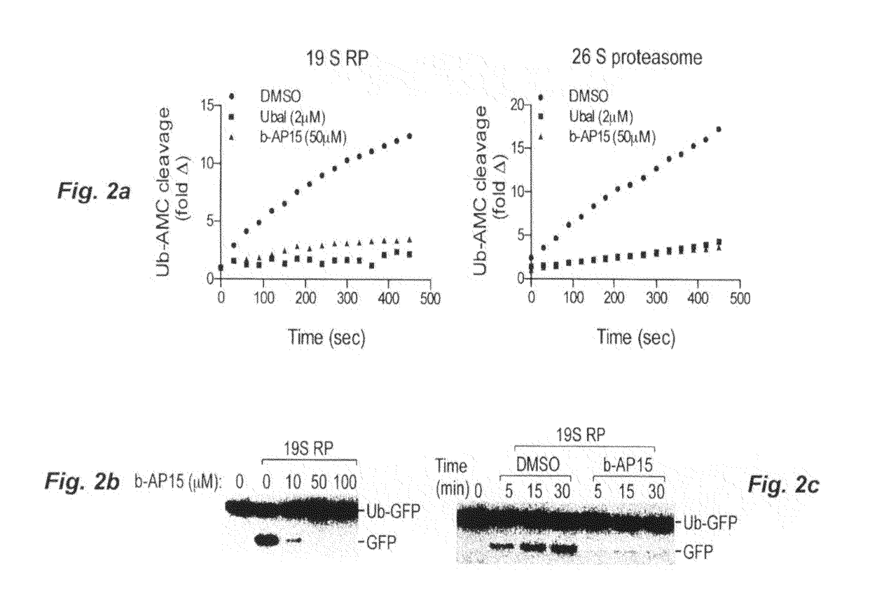

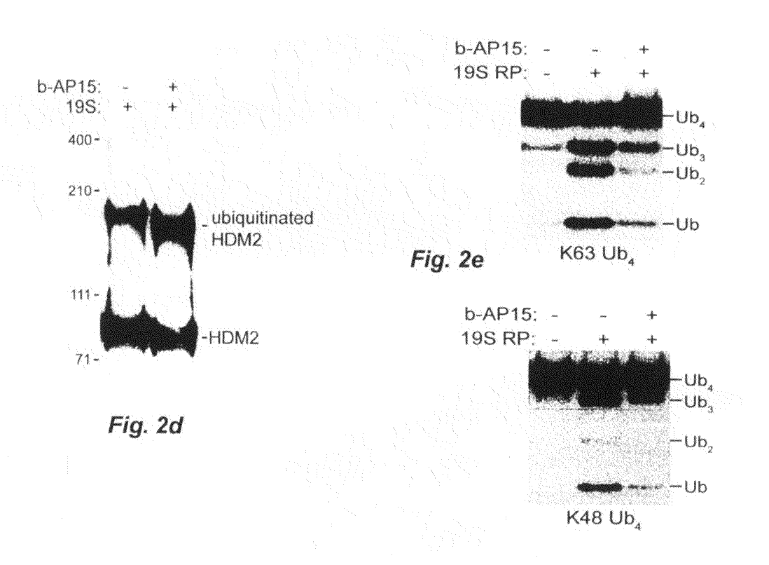

Inhibition of Ub-AMC cleavage by 19S RP or 26S proteasomes following treatment with b-AP15; ubiquitin aldehyde (Ubal), a general DUB inhibitor, was included as a control (FIG. 2a). Immunoblot of 19S RP mediated cleavage of Ub-GFP (FIG. 2b); 19S RP were pre-treated with DMSO or indicated concentrations of b-AP15 followed by addition of recombinant Ub-GFP as a DUB substrate. Kinetics of 19S RP Ub-GFP cleavage following b-AP15 (50 μM) treatment (FIG. 2c). b-AP15 inhibits de-ubiquination of Hdm2 (FIG. 2d); ubiquinated Hdm2 was added to DMSO or b-AP15 (50 μM) treated 19S RP followed by immunoblotting. Ubiquitin chain disassembly reactions of K63/K48 linked ubiquitin tetramers by 19S RP following treatment with DMSO or b-AP15 (50 μM) (FIG. 2e).

### Example 3

### b-AP15 Inhibits the 19S RP DUBs UCHL5 and USP14

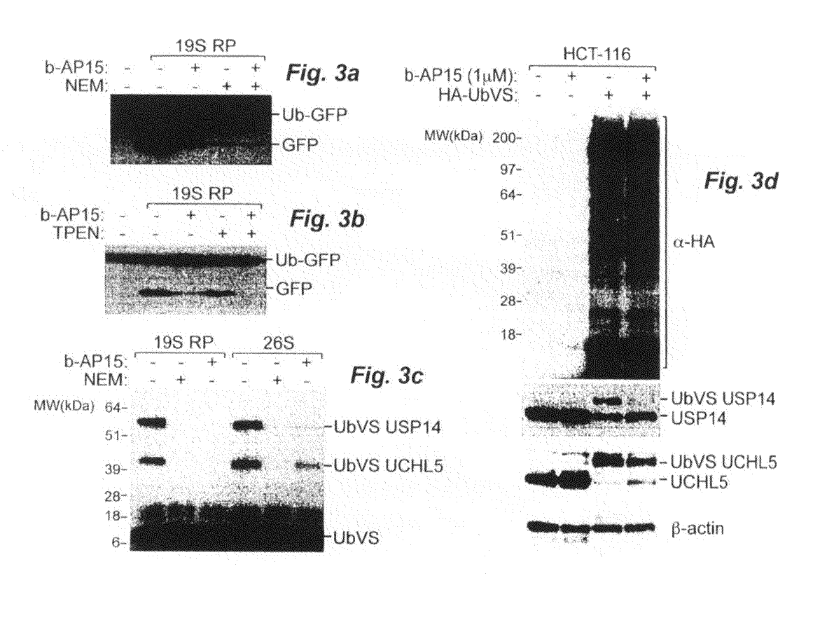

19S RP were pre-treated with DMSO, NEM (10 mM) b-AP15 (50 μM) (FIG. 3a) or TPEN (250 μM) (FIG. 3b) followed by addition of Ub-GFP and immunoblotting with anti-GFP antibodies. Active site directed labelling of proteasomal DUBs (FIG. 3c); purified 19S or 26S proteasomes were pre-treated with DMSO, NEM or bAP15 followed by labeling with HA-UbVS and immunoblotting. Immunoblot of HCT116 cells treated with b-AP15 (1 μM) for 3 h (FIG. 3d); DUBs from whole cell lysates were labelled with HA-UbVS followed by SDS-PAGE and immunoblotting with indicated antibodies.

### Example 4

### b-AP15 Inhibits Tumor Growth In Vivo

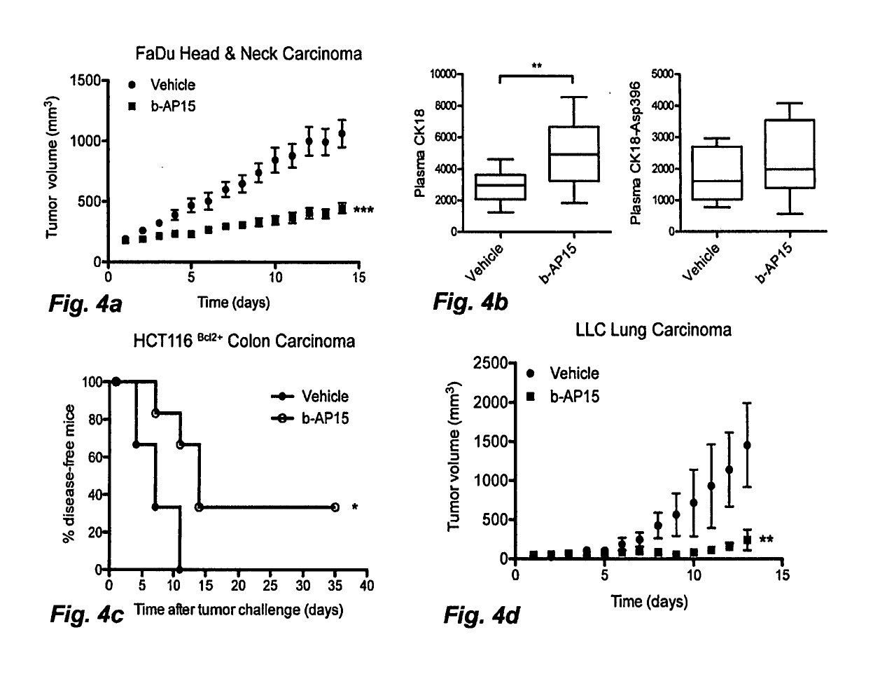

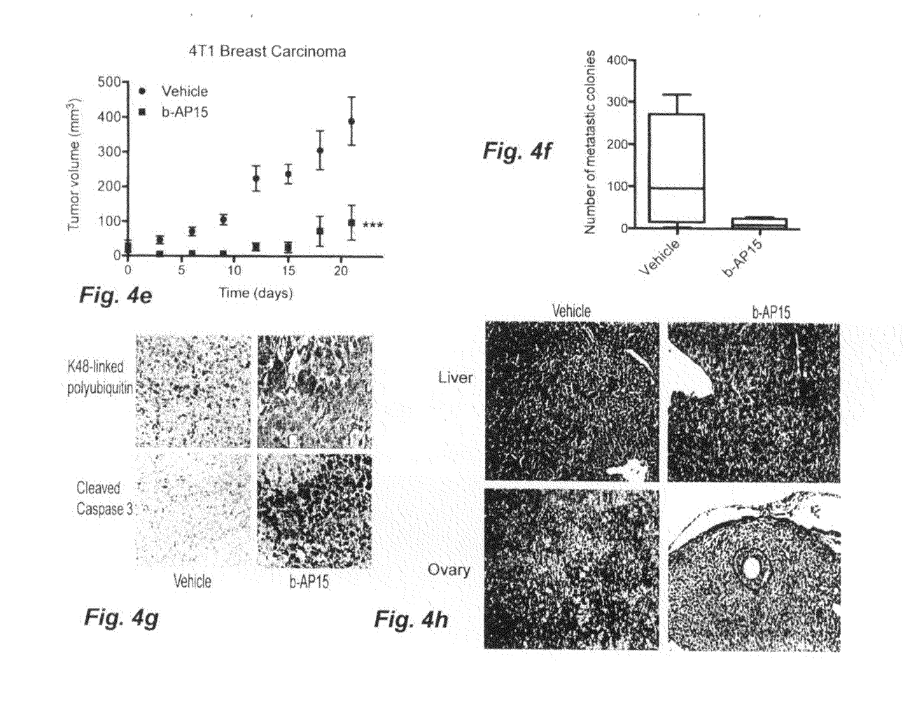

SCID mice bearing FaDu human tumor xenografts were randomized at tumor take (200 mm³) and treated by daily subcutaneous injection with either vehicle (n=10) or 5 mg kg⁻¹ b-AP15 (n=15) for 10 days. Mean tumor volume±SEM shown (***P=<0.001) (FIG. 4a). Total levels of tumor derived CK18 and caspase cleaved (CK 18-Asp396) in circulation following b-AP15 treatment (**P=0.01) (FIG. 4b). Disease free survival of nude mice challenged with HCT-116^{Bel-2+} cells (FIG. 4c). Mice were treated with vehicle (n=6) or 5 mg kg⁻¹ b-AP15 (n=6) 4-5 times weekly for 3 weeks and monitored for tumor onset (log-rank, P=0.0136, hazard ratio=7.9). C57BL/6J mice bearing syngenic lung carcinoma (LLC) tumors were treated with either vehicle (n=4) of 5 mg kg⁻¹ b-AP15 (n=4) in a one day on/two days off cycle (FIG. 4d); mean tumor volume±SEM shown (P=<0.01). BALB/c mice bearing orthotopic breast carcinomas (4T1) were treated with either vehicle (n=5) or 2.5 mg kg⁻¹ b-AP15 (n=5) in a one day on/three days off cycle (FIG. 4e); mean tumor volume±SEM shown (**P=<0.01). Box and whisker plots of pulmonary metastatic colonies from vehicle or b-AP15 treated 4T1 breast carcinomas (FIG. 4f); boxes represent upper and lower quartiles and median, whiskers show maximum and minimum values. Representative immunohistochemical staining for K48-linked ubiquitin accumulation and cleaved caspase-3 in vehicle and b-AP15 treated 4T1 tumors, original magnification ×20 (FIG. 4g). AML infiltration in liver and ovary of vehicle and b-AP15 treated mice (FIG. 4h). Liver of vehicle treated mice showed invasion of leukemic blasts along with glycogen depletion and non-specific hemorrage. Ovary section of vehicle treated mice showed massive invasion of leukemic blasts and interstitial bleeding. In contrast, liver and ovary from b-AP15 treated mice showed few infiltrated blasts and normal morphology (original magnification ×20).

### Example 5

### b-AP15 does not Induce DNA Damage

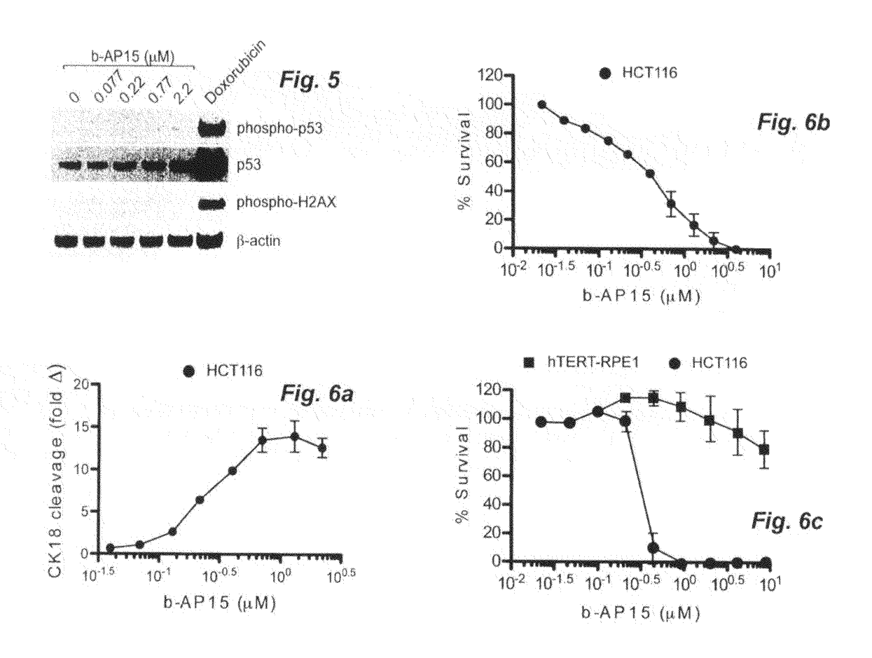

HCT-116 cells were treated with b-AP15 or doxorubicin (100 nM, as a positive control for genotoxic stress for 18 h) (FIG. 5). Cell lysates were immuno-blotted with antibodies for phosphorylated p53 and histone H2 Ax marker for DNA damage of for total levels of p53 and β-actin as loading controls.

### Example 6

### b-AP15 Induces Apoptosis and Inhibits Cell Survival of HCT-116 Cells Whereas PBMC (Peripheral Blood Mononuclear Cells) and Immortalized hTERT-RPE1 are Less Sensitive

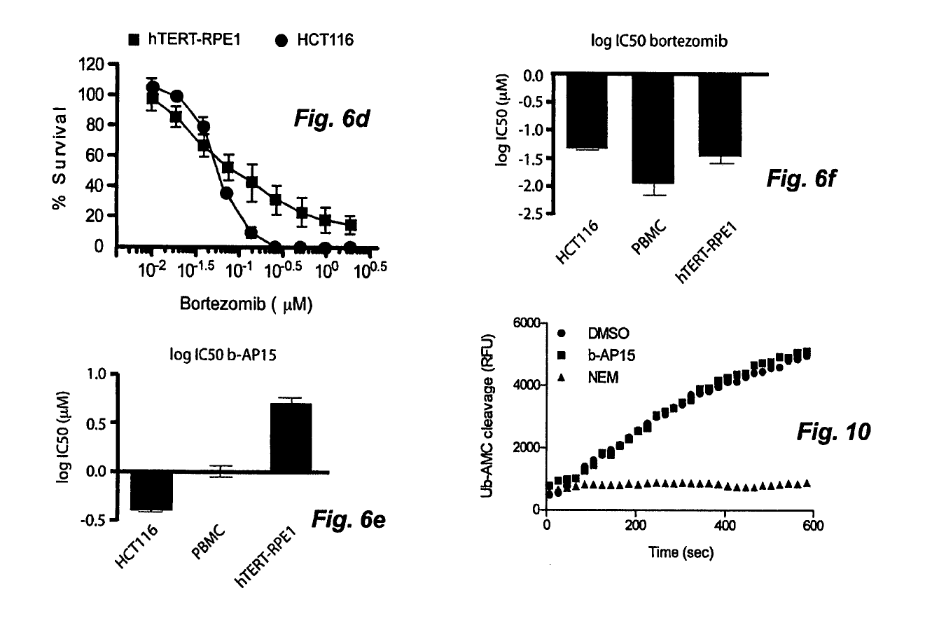

HCT-116 cells were treated with increasing concentrations of b-AP15 for 24 h and the levels of apoptosis were determined by measuring the levels of caspase cleaved cytokeratin-18 (CK18) by ELISA assay (FIG. 6a). HCT-116 cells were treated with increasing concentrations of b-AP15 for 48 h). Cell viability was determined by acid-phosphatase activity assay. Mean values±s.d. shown (FIG. 6b). HCT-116 or hTERT-REP1 cells were treated with increasing concentrations of b-AP15 for 72 h followed by analysis of cytotoxicity using the FMCA method (44) (FIG. 6c). HCT-116 or hTERT-REP1 cells were treated with increasing concentrations of bortezomib for 72 h followed by analysis of cytotoxicity using the FMCA method (FIG. 6d). HTERT-RPE1 is an immortalized human retinal pigment epithelial cell line (29). IC50 was determined from log concentration/effect curves in Graph Pad Prism (GraphPad Software Inc. CA, USA) using non-linear regression analysis (four parameter model with variable Hill slope) (FIGS. 6e, 6f). Concentration/response curves were generated in two-fold dilutions at eight concentrations of b-AP15 and bortezomib in triplicate using the FMCA assay. The results are expressed as log IC50+SD from four or five independent experiments (HCT-116, n=5; PBMC, n=4; hTERT-RPE1, n=5).

### Example 7

### Dose Response Curves of Apoptosis Induction in Isogenic Clones of Hct-116 Cells

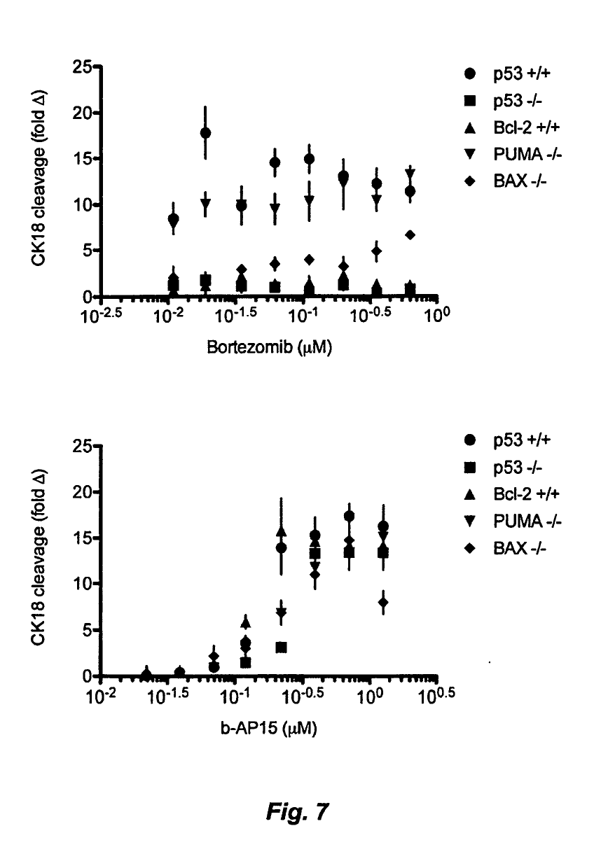

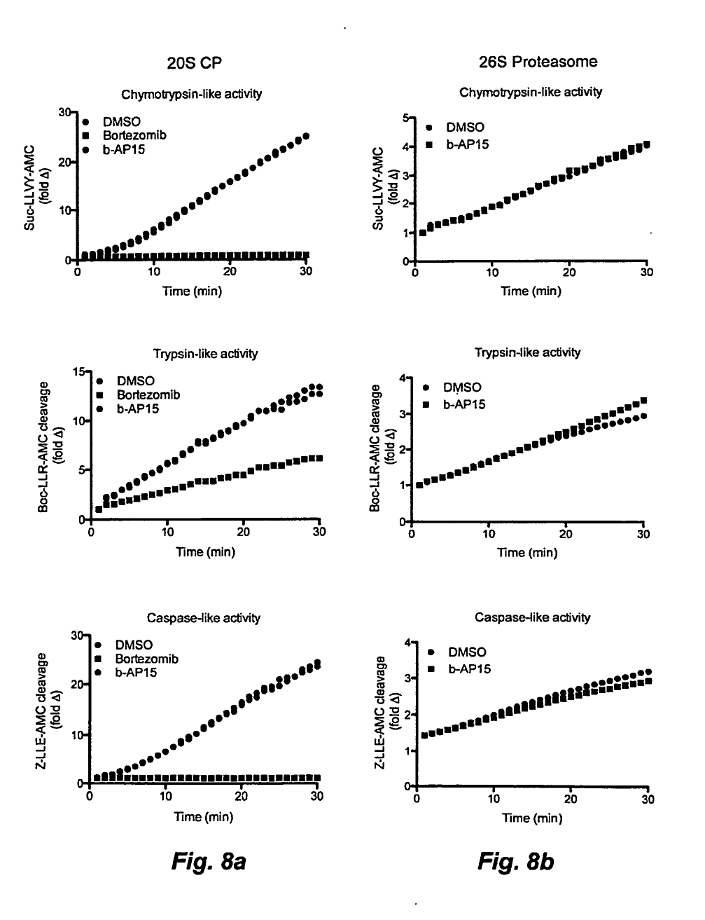

HCT-116 cells were treated with increasing concentrations of bortezomib or b-AP15 for 24 h and the levels of apoptosis were determined by measuring the levels of caspase cleaved cytokeratin-18 (CK-18) by ELISA assay (Mean fold change±s.d, n=4) (FIG. 7).

### Example 8

### b-AP15 Does not Inhibit the Proteolytic Activities of the Proteasome

20S CP (2 nM) was pretreated with DMSO, b-AP15 (50 μM) or bortezomib (100 nM) for 5 min in assay buffer (25 mM HEPES, 0.5 mM EDTA, 0.03% SDS) followed by the addition of 100 μM of the fluorogenic substrates Suc-LLVY-AMC, Z-LLE-AMC or Boc-LRR-AMC for analysis of proteasome chymotrypsin-like, caspase-like and trypsin-like activities, respectively (FIG. 8a). 26S proteasomes (2 nM) in assay buffer (25 mM HEPES, 50 mM NaCl, 1 mM MgCl₂, 2 mM ATP, 1 mM DTT) were treated as in the experiment illustrated in FIG. 8a (FIG. 8b). Values represent fold cleavage in relative fluorescent units.

### Example 9

### b-AP15 Does not Cause Dissociation of 19S and 20S Particles or Alter Ubiquitin Binding

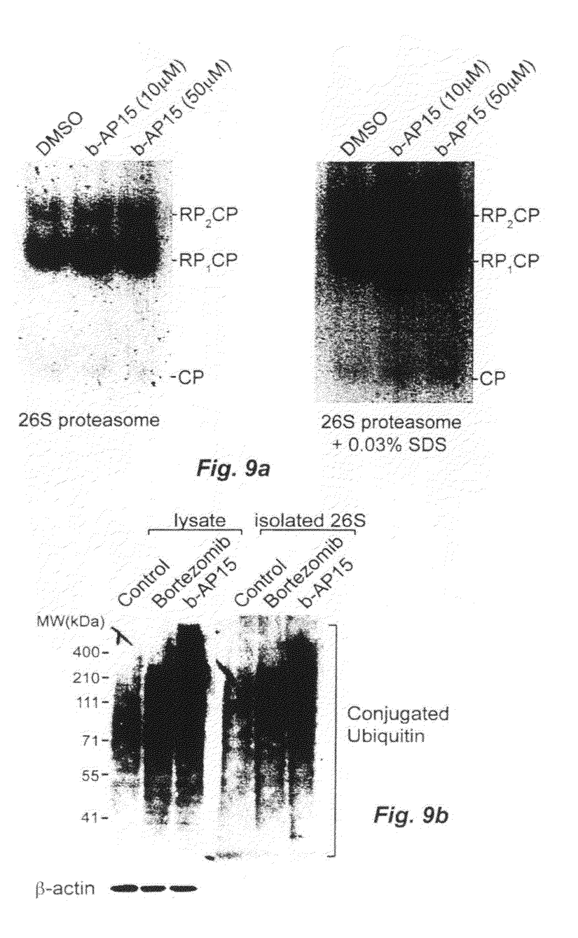

Substrate overlay assay of b-AP15 treated proteasomes (FIG. 9a). Purified 26 S proteasome was treated with b-AP15 (10 μM or 50 μM) separated by native gel electrophoresis and assayed for proteolytic activity using Suc-LLVY-AMC as a fluorogenic substrate for peptidase activity. Analysis of the gels showed the presence of doubly (RP₂CP) and singly (RP₁CP) capped proteasomes in both control and b-AP15 lanes. The addition of 0.03% SDS did not reveal an increase in the presence of uncapped 20S core particles. b-AP15 does not alter proteasome-ubiquitin binding activity (FIG. 9b). HCT-116 cells were treated with bortezomib (100 nNM) or b-AP15 (1 μM), and the proteasomes were affinity purified. Levels of associated polyubiquitin were determined by immunoblotting.

### Example 10

### b-AP15 is not a General DUB Inhibitor

HTC-116 cells were treated for 3 h with b-AP15 (1 μM) (FIG. 10). Lysates treated with 10 mM N-ethylmaleimide (NEM) were included as a control for total DUB inhibition. DUB activity was determined from cell lysates by measuring cleavage of the fluorogenic substrate ubiquitin-7-amido-4-methylcoumarin (Ub-AMC).

### Example 11

### Biochemical Characterization of b-AP15 Binding

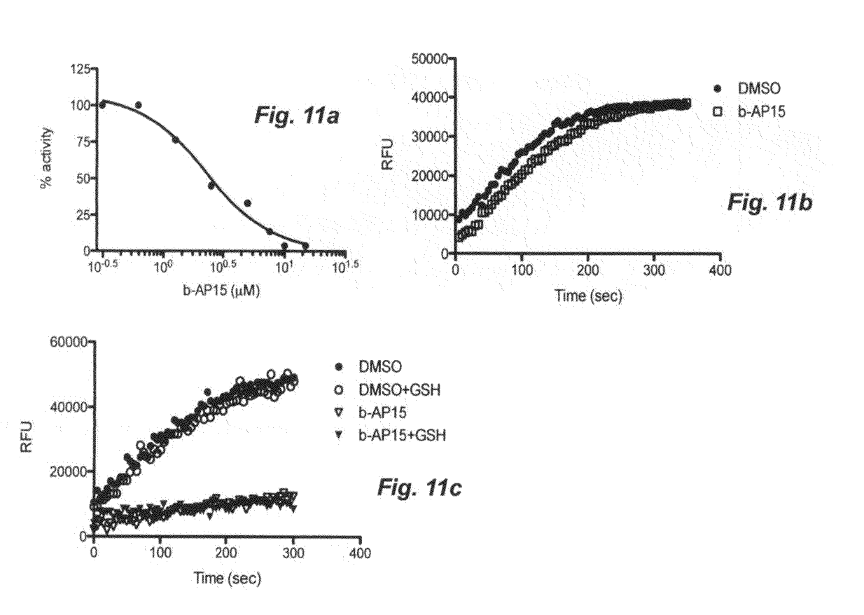

Dose response of b-AP15 (FIG. 11a): Purified 10S proteasomes (5 nM) were treated with indicated concentrations of b-AP15, and DUB activity was determined by detection of Ub-AMC cleavage. The IC50 value (2.1±0.411 μM) was determined from log concentration curves in Graph Pad Prism using non-linear regression analysis (mean values±SD, n=3). It should be noted that IC50 observed in cell-free assays is somewhat higher than that observed in cells, probably due to the hydrophobicity of b-AP15 (X Log P=3.3) resulting in enrichment of the compound in cells (11). Reversibility of b-AP15 inhibition (FIG. 1b): The reversibility of inhibition was determined by measuring recovery of DUB activity after rapid dilution of the enzyme/b-AP15 complex. A reaction mix containing 50 times the 19S concentration normally used in reactions (250 mM) and 10 times the calculated IC50 value for b-AP15 (25 μM) was incubated on ice for 15 min followed by a 50-fold dilution in reaction buffer to give a final concentration of 5 nM for 19′S and 0.5 μM for b-AP15. The linear reaction curves of Ub-AMC cleavage show that b-AP15 is a reversible inhibitor. Determination of whether b-AP15 reacts non-specifically with cysteine residues (FIG. 11c). 19S (5 nM) was treated with b-AP15 (10 μM) or b-Ap15 (10 μM) mixed with reduced glutathione (GSH (2 mM). The presence of glutathione did not reduce b-AP15 mediated inhibition of 19S DUB activity.

### Example 12

### b-AP15 is not a General DUB Inhibitor

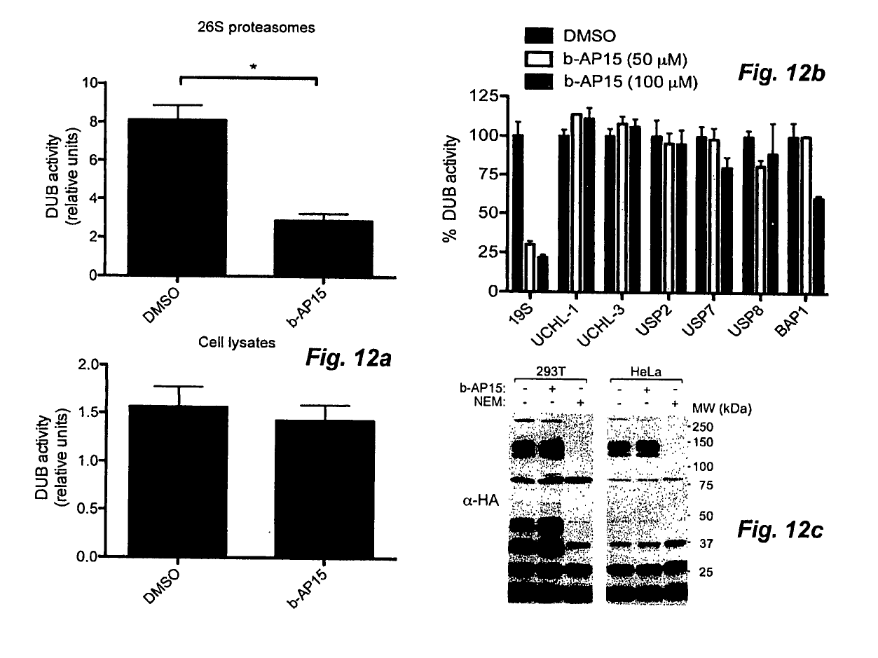

HCT-116 cells were treated for 3 h with b-AP15 (1 μM) and the proteasomes were affinity purified (FIG. 12a). Proteasome DUB activity is expressed as cleavage of Ub-AMC/suc-LLVY-AMC to normalize for proteasome recovery (P=0.012, unpaired t-test, two tailed). b-AP15 does not inhibit non-proteasomal DUBs (FIG. 12b). Recombinant non-proteasomal DUBs were treated with b-AP15 and % activity determined. Cell lysates from 293T and HeLa cells were treated with b-AP15 (50 μM) followed by active labelling with HA-UbVS (FIG. 12c). All samples were run on SD-PAGE followed by immunoblotting with α-HA antibodies.

### Example 13

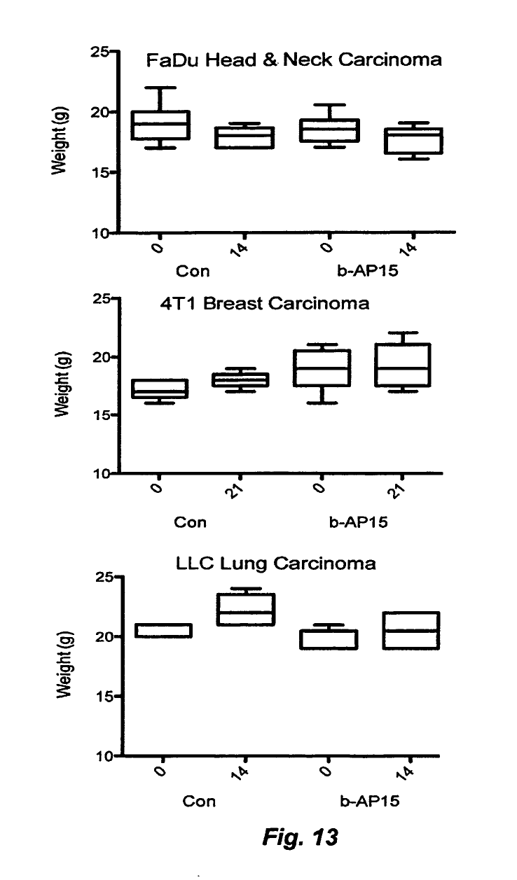

### b-AP15 Treatment does not Significantly Alter Animal Weight (FIG. 13)

The difference in weight at the start and the endpoint between control and treated animals for the xenografts shown in FIG. 4 was: FaDu, −1.3%; LLC, +2.1%; 4T1+5.8%. Boxes represent the upper and lower quartiles and median, whiskers show maximum and minimum values.

### Example 14

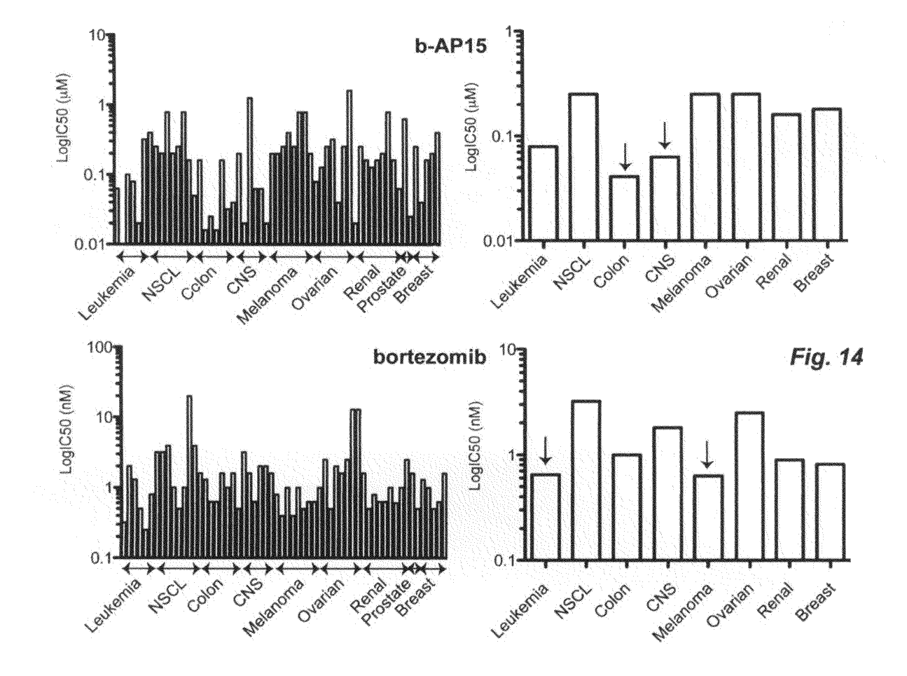

### Sensitivity of Cell Lines in the NC160 Cell Line to b-AP15 and Bortezomib (FIG. 14)

Shown are IC50 values for individual cell lines (left hand graphs) and median IC50 for each tumor type (right hand graphs). Data have been taken from www.dtp.nci.nih.gov. Arrows indicate the two most sensitive tumor cell types for each drug.

### Example 15

### Expression of Chaperone Genes Observed in bAP15-Treated Cells

Expression of chaperone genes observed in bAP15-treated cells (Table 1) is indicative of induction of a proteotoxic response.

TABLE 1
Induction of chaperone expression after b-AP15 treatment

| | | | Expression | | |
|---|---|---|---|---|---|
| Probe Set | | Gene | values | | Fold |
| ID | Gene Title | Symbol | b-AP15 | Vehicle | change |
| 117_at | heat shock 70 kDa protein 6 (HSP70B′) | HSPA6 | 24149 | 33 | 725 |
| 225061_at | DnaJ (Hsp40) homolog, A4 | DNAJA4 | 21103 | 711 | 30 |
| 203810_at | DnaJ (Hsp40) homolog, B4 | DNAJB4 | 1955 | 123 | 16 |
| 205543_at | heat shock 70 kDa protein 4-like | HSPA4L | 5452 | 406 | 13 |
| 200666_s_at | DnaJ (Hsp40) homolog, B1 | DNAJB1 | 33900 | 5251 | 6 |
| 241716_at | heat shock 60 kDa protein 1 | HSPD1 | 487 | 77 | 6 |
| 203811_s_at | DnaJ (Hsp40) homolog, B4 | DNAJB4 | 960 | 178 | 5 |
| 202581_at | heat shock 70 kDa protein 1B | HSPA1B | 31068 | 6382 | 5 |
| 206976_s_at | heat shock 105 kDa/110 kDa protein 1 | HSPH1 | 40974 | 8427 | 5 |
| 211016_x_at | heat shock 70 kDa protein 4 | HSPA4 | 1803 | 422 | 4 |
| 202843_at | DnaJ (Hsp40) homolog, B9 | DNAJB9 | 1879 | 449 | 4 |
| 200880_at | DnaJ (Hsp40) homolog, A1 | DNAJA1 | 19970 | 4872 | 4 |
| 200800_s_at | heat shock 70 kDa protein 1A | HSPA1A | 57478 | 14352 | 4 |

Further analysis by quantitative PCR showed that b-AP15 induces a stronger HSPA6 (Hsp70B′), HSPA1B and DNAJB1 (Hsp40) expression than bortezomib (Table 2). HSPA6, which is known to be induced in response to accumulation of damaged proteins (35), was induced >1000-fold by b-AP15. These findings indicate that high molecular weight ubiquitin substrate complexes accumulating as a result of DUB inhibition can generate strong cytotoxicity that is insensitive to Bcl-2 over-expression.

TABLE 2
Quantitation of chaperone gene induction.

| | Gene | Fold induction^{#} | |
|---|---|---|---|
| Gene Title | Symbol | b-AP15 | bortezomib |
| heat shock 70 kDa protein 6 | HSPA6 | 1550 | 60 |
| (Hsp70B′) | | | |
| heat shock 70 kDa protein 1B | HSPA1B | 21 | 12 |
| (Hspa1b) | | | |
| DnaJ homolog, B1 (Hsp40B1) | DNAJB1 | 22 | 5 |

^{#}HCT116 cells were treated with IC90 concentrations of b-AP15 or bortezomib and mRNA levels were determined after reverse transcription and real time PCR, Fold induction is expressed as fold untreated control. The experiment was repeated with similar results.

The cellular response to b-AP15 is not only distinct from that of bortezomib in regard of involvement of apoptosis regulators but also in regard of the sensitivity of tumor cell lines in the NCI-60 cell line panel (http://dtp.nci.nih.gov). Inhibitors of 19S RP DUB activity should display a therapeutic spectrum different from that of inhibitors of 20S enzymatic activity, and therefore expand the arsenal of therapy options in oncology.

### Example 16

### Synthesis of b-AP15

### (3E,5E)-3,5-bis[(4-nitrophenyl)methylidene]piperidin-4-one

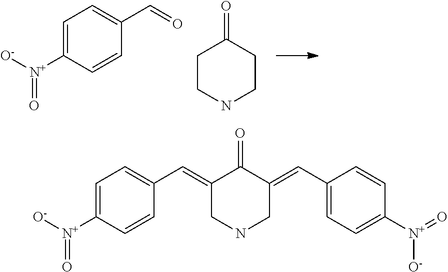

4-Nitrobenzaldehyde (12.39 g, 82 mmol) and 4-piperidone.HCl (6.14 g, 40 mmol) were suspended in acetic acid (27.5 mL). Hydrochloric acid gas generated by dropwise addition of sulfuric acid (400 mmol) on sodium chloride (400 mmol) was bubbled through the reaction mixture, followed by conc. sulfuric acid (0.5 mL) and stirring overnight (16 h) at room temperature. LCMS analysis showed almost complete conversion to product. The reaction mixture was filtered and the collected solid suspended in sat K₂CO₃ (80 mL) acetone (80 mL). The pH was adjusted to 12 with saturated aqueous Na₂CO₃ and the mixture stirred for 30 min at room temperature. The slurry was filtered and washed with water (160 mL in small portions) to give a fine yellow powder. Drying overnight under vacuum in a desiccator gave 7.81 g (3E,5E)-3,5-bis[(4-nitrophenyl)methylidene]piperidin-4-one (53%), >98% pure according to LCMS (ACE C8, 3.0 μm, 50×3.0 mm, 10% to 97% acetonitrile in 3 min, 1 ml/min, detection at 305±90 nm).

### (3E,5E)-3,5-bis[(4-nitrophenyl)methylidene]-1-(prop-2-enoyl)piperidin-4-one (b-AP15)

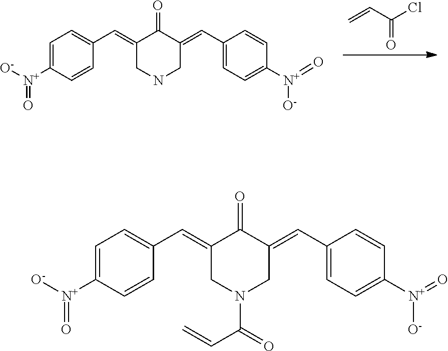

K₂CO₃ (4.0 g, 29 mmol) was dissolved in water (10 mL) and mixed with acetone (10 mL). To the clear 2-phasic liquid system cooled on an ice-bath was added (3E,5E)-3,5-bis[(4-nitrophenyl)methylidene]piperidin-4-one (2.1 g, 5.75 mmol). Over a few minutes acrylic acid chloride (0.80 g, 0.715 mL, 8.84 mmol) was added dropwise to the 2-phasic system. The reaction mixture was stirred at 0° C. for 10 min, then for 1 h at room temperature. The reaction vessel was cooled in an ice-bath, and more acrylic acid chloride (0.40 g, 0.358 mL, 4.42 mmol) was added dropwise. The reaction mixture was allowed to warm to room temperature and was stirred for 3 h. Workup by adding 20 mL water, filtering and washing with 20 mL water rendered a solid residue, which was purified on silica with a gradient of 0-5% acetone in dichloromethane. Solvent was evaporated from the combined pure fractions to 1.12 g (46%) yellow solid b-AP15. Basic (Xbridge C18, 3.5 μm, 50×3.0 mm, 10% to 97% MeCN in 10 mM NH₄HCO₃ (pH10) over 3 min, 1 mL/min, detection at 305±90 nm) and acidic (ACE C8, 3.0 μm, 50×3.0 mm, 10% to 97% acetonitrile in 0.1% aqueous TFA over 3 min, 1 ml/min, detection at 305±90 nm) LCMS methods showed >98% purity. Melting point 170-172° C.

### Example 17

### Pharmaceutical Composition A (Aqueous Suspension)

b-AP15 (25.2 mg) is dissolved in 1 ml of dimethyl sulfoxide. The solution is added dropwise to 10 ml of vigorously stirred saline. The formed suspension, which can be stabilized by adding 1% by weight of PVP, can be used for intramuscular, intravenous or subcutaneous administration.

### Example 18

### Pharmaceutical Composition B (Tablet)

Tablets for oral administration are produced by blending 2.0 g of b-AP15 (powder, <10 mu, 90%) with microcrystalline cellulose (1.30 g), corn starch (0.50) g, silica (0.20) g, Mg stearate (0.12 mg). The mixture is dry compressed to 400 mg tablets, which are sugar coated.

### Example 19

### Pharmaceutical Composition C (Solution)

b-AP15 (14 mg) was dissolved in 0.5 ml of Cremophor EL (BASF Corp.) and absolute ethanol was added to 1.0 ml. The clear solution was filled into glass vials for injection.

### REFERENCES

1. Masdehors, P et al., Increased sensitivity of CLL-derived lymphocytes to apoptotic death activation by the proteasome-specific inhibitor lactacystin. Br J Haematol 105, 752-757, doi:bjh1388 [pii] (1999).
2. DeMartino, G N et al., PA700, an ATP-dependent activator of the 20 S proteasome, is an ATPase containing multiple members of a nucleotide binding protein family. J Biol Chem 69, 20878-20884, http://www.ncbi.nlm.nih.gov/entrez/query.fcgi?cmd=Retrieve&db=PubMed &dopt=Citation&list_uids=8063704 (1994) (1994).
3. Rechsteiner, M et al., The multicatalytic and 26 S proteases. J Biol Chem 268, 6065-6068, http://www.ncbi.nlm.nih.gov/entrez/query.fcgi?cmd=Retrieve&db=PubMed&dopt=Citation&list_uids=8454582 (1993).
4. Adams, J & Kauffman, M, Development of the proteasome inhibitor Velcade (Bortezomib). Cancer Invest 22, 304-311, http://www.ncbi.nlm.nih.gov/entrez/query.fcgi? cmd=Retrieve &db=PubMed&dopt=Citation&list_uids=15199612 (2004).
5. Erdal, H et al., Induction of lysosomal membrane permeabilization by compounds that activate p53-independent apoptosis. Proc Natl Acad Sci USA 102, 192-197, doi:0408592102 [pii]10.1073/pnas.0408592102 (2005).
6. Berndtsson, M et al., Induction of the lysosomal apoptosis pathway by inhibitors of the ubiquitin-proteasome system. Int J Cancer 124, 1463-1469, doi:10.1002/ijc.24004 (2009).
7. Lamb, J et al., The Connectivity Map: using gene-expression signatures to connect small molecules, genes, and disease. Science 313, 1929-1935, doi:313/5795/1929 [pii]10.1126/science.1132939 (2006).
8. Adams, J et al., Potent and selective inhibitors of the proteasome: dipeptidyl boronic acids. Bioorg Med Chem Lett 8, 333-338 333-338, doi:50960894×98000298 [pii] (1998).
9. Shibata, T et al., An endogenous electrophile that modulates the regulatory mechanism of protein turnover: inhibitory effects of 15-deoxy-Delta 12,14-prostaglandin J2 on proteasome. Biochemistry 42, 13960-13968, doi:10.1021/bi035215a (2003).
10. Yang, H et al., Celastrol, a triterpene extracted from the Chinese “Thunder of God Vine,” is a potent proteasome inhibitor and suppresses human prostate cancer growth in nude mice. Cancer Res 66, 4758-4765 4758-4765, doi:66/9/4758 [pii]10.1158/0008-5472.CAN05-4529 (2006).
11. Yang, H et al., The tumor proteasome is a primary target for the natural anticancer compound Withaferin A isolated from “Indian winter cherry”. Mol Pharmacol 71, 426-437, doi:mol.106.030015 [pii]10.1124/mol.106.030015 (2007).
12. Menendez-Benito, V et al., Endoplasmic reticulum stress compromises the ubiquitin-proteasome system. Hum Mol Genet. 14, 2787-2799, doi:ddi312 [pii]10.1093/hmg/ddi312 (2005).
13. Mullally, J E & Fitzpatrick, F A, Pharmacophore model for novel inhibitors of ubiquitin isopeptidases that induce p53-independent cell death. Mol Pharmacol 62, http://www.ncbi.nlm.nih.gov/entrez/query.fcgi?cmd=Retrieve&db=PubMed& dopt=Citation&list_uids=12130688 (2002).
14. Glickman, M H & Ciechanover, A, The ubiquitin-proteasome proteolytic pathway: destruction for the sake of construction. Physiol Rev 82, 373-428, doi:10.1152/physrev.00027.2001 (2002).
15. Guterman, A & Glickman, M H, Complementary roles for Rpn11 and Ubp6 in deubiquitination and proteolysis by the proteasome. J Biol Chem 279, 17291738, doi:10.1074/jbc.M307050200 [pii] (2004).
16. Hofmann, R M & Pickart, C M et al., Noncanonical MMS2-encoded ubiquitin conjugating enzyme functions in assembly of novel polyubiquitin chains for DNA repair. Cell 96, 645-653, doi:S0092-8674(00)80575-9 [pii] (1999).
17. Vong, Q P et al., Chromosome alignment and segregation regulated by ubiquitination of surviving cells. Science 310, 1499-1504, doi:310/5753/1499 [pii]10.1126/science.1120160 (2005).
18. Borodovsky, A et al., A novel active site-directed probe specific for deubiquitylating enzymes reveals proteasome association of USP14. EMBO J 20, 5187-5196, doi:10.1093/emboj/20.18.5187 (2001).
19. Lam, Y A et al., Specificity of the ubiquitin isopeptidase in the PA700 regulatory complex of 26 S proteasomes. J Biol Chem 272, 28438-28446, http://www.ncbi.nlm.nih.gov/entrez/query.fcgi?cmd=Retrieve&db=PubMed&dopt=Citation&list_uids=9353303 (1997).
20. Verma, R et al., Role of Rpn11 metalloprotease in deubiquitination and degradation by the 26S proteasome. Science 298, 611-615, doi:10.1126/science.10758981075898 [pii] (2002).
21. Yao, T & Cohen, R E, A cryptic protease couples deubiquitination and degradation by the proteasome. Nature 419, 403-407, doi:10.1038/nature01071nature01071 [pii] (2002).
22. Kramer, G et al., Differentiation between cell death modes using measurements of different soluble forms of extracellular cytokeratin 18. Cancer Res 64, 1751-1756 (2004) http://www.ncbi.nlm.nih.gov/entrez/query.fcgi?cmd=Retrieve&db=PubMed& dopt=Citation&list_uids=14996736 (2004).
23. Olofsson, M H et al., Specific demonstration of drug-induced tumour cell apoptosis in human xenograft models using a plasma biomarker. Cancer Biomarkers 5, 117-125, http://www.ncbi.nlm.nih.gov/pubmed/19407366 (2009).
24. Reyes-Turcu, F E et al., Regulation and cellular roles of ubiquitin-specific deubiquitinating enzymes. Annu Rev Biochem 78, 363-397, http://www.ncbi.nlm.nih.gov/entrez/query.fcgi?cmd=Retrieve&db=PubMed&dopt=Citation&list_uids=19489724 (2009).
25. Koulich, E et al., Relative structural and functional roles of multiple deubiquitylating proteins associated with mammalian 26S proteasome. Mol Biol Cell 19, 1072-1082, doi:E07-10-1040 [pii]10.1091/mbc.E07-10-1040 (2008).
26. Bunz, F. et al., Requirement for p53 and p21 to sustain G2 arrest after DNA damage. Science 282, 1497-1501 (1998).
27. Pietenpol, J A et al., Paradoxical inhibition of solid tumor cell growth by bcl2. Cancer Res 54, 3714-3717 (1994).
28. Menendez-Benito, V et al., Endoplasmic reticulum stress compromises the ubiquitin-proteasome system. Hum Mol Genet. 14, 2787-2799, doi:ddi312 [pii]10.1093/hmg/ddi312 (2005).
29. Bodnar, A G et al., Extension of life-span by introduction of telomerase into normal human cells. Science 279, 349-352 (1998).
30. Lamb, J et al., The Connectivity Map: using gene-expression signatures to connect small molecules, genes, and disease. Science 313, 1929-1935, doi:313/5795/1929 [pii] 10.1126/science.1132939 (2006).
31. Elsasser, S et al., Characterization of the proteasome using native gel electrophoresis. Methods Enzymol 398, 353-363, doi:S0076-6879(05)98029-4 [pii]10.1016/50076-6879(05)98029-4 (2005).
32. Guterman, A & Glickman, M H, Complementary roles for Rpn11 and Ubp6 in deubiquitination and proteolysis by the proteasome. J Biol Chem 279, 1729-1738, doi:10.1074/jbc.M307050200M307050200 [pii] (2004).
33. Hagg, M et al., A novel high-through-put assay for screening of pro-apoptotic drugs. Invest New Drugs 20, 253-259 (2002).
34. Lindhagen, E et al., The fluorometric microculture cytotoxicity assay. Nat Protoc 3, 1364-1369, doi:nprot.2008.114 [pii]10.1038/nprot.2008.114 (2008).
35. Pulaski, B A & Ostrand-Rosenberg, S, Mouse 4T1 breast tumor model. Curr Protoc Immunol Chapter 20, Unit 20 22, doi:10.1002/0471142735.im2002s39 (2001).

1. A method of treating in a person a cancer tumor refractory to treatment with bortezomib or an agent sharing the apoptosis generating activity of bortezomib or any other anti-cancer drug, comprising administering, in a pharmaceutically acceptable carrier, a pharmacologically effective dose of an agent selected from the group consisting of b-AP15 and other proteasome inhibitor abrogating the deubiquitinating (DUB) activity of the 19S RP DUBs.

2. The method of claim 1, wherein cells of the tumor are refractory to treatment due to over-expression of the intrinsic apoptosis-inhibitor Blc-2

3. The method of claim 1 or 2, wherein 19S RP DUBs comprise UCHL5 and USP14.

4. The method of claim 1, wherein the deubiquitinating (DUB) activity of non-proteasomal DUBs is not affected.

5. The method of claim 1, wherein the agent is dissolved or suspended in an liquid carrier.

6. The method of claim 5, wherein administration is by intravenous, intramuscular or subcutaneous injection or infusion.

7. The method of claim 1, wherein administration is peroral.

8. The method of claim 7, wherein the carrier is a tablet or capsule.

9. The method of any of claims 1-8, wherein the pharmacologically effective dose is from 0.01 g/kg body weight to 0.1 g or 1.0 g or more/kg body weight, consideration being given to whether the agent is administered systemically or locally.

10. The method of any of claims 1-8, wherein “refractory to treatment” signifies that treatment of a cancer with a single dose of bortezomib or an agent sharing the apoptosis generating activity of bortezomib or any other anti-cancer drug does not substantially reduce the growth rate per month of the cancer observed immediately prior to the treatment, such by more than 25 percent or by more than 10 percent or by more than 5 percent.

11. The method of any of claims 1-10, wherein the cancer is selected from multiple myeloma, breast cancer, ovary cancer, lung cancer, colon cancer, prostate cancer, pancreas cancer.

12. The method of any of claims 1-11, comprising selecting a person to be treated by determining the growth rate of the cancer prior to and upon administration of bortezomib or said active principle sharing the mechanism of deubiquitinating activity inhibition of bortezomib or said other anti-cancer drug, a positive growth rate, in particular a growth rate of more than 5% or more than 10% or more than 25% per month constituting a selection marker.
